(19) United States
(12) Patent Application Publication
(10) Pub. No.: US 2015/0173648 A1
(43) Pub. Date: Jun. 25, 2015

(54) HEALTH SUPPORT DEVICE, METHOD, AND RECORDING MEDIUM STORED WITH PROGRAM

(71) Applicant: TANITA CORPORATION, Tokyo (JP)

(72) Inventors: Ayumi SANO, Tokyo (JP); Naotaka MINAGAWA, Tokyo (JP); Miyuki KODAMA, Tokyo (JP); Yasuhiro KASAHARA, Tokyo (JP)

(73) Assignee: TANITA CORPORATION, Tokyo (JP)

(21) Appl. No.: 14/564,206

(22) Filed: Dec. 9, 2014

(30) Foreign Application Priority Data
Dec. 19, 2013 (JP) 2013-262935

(51) Int. Cl. A61B 5/08; G01N 33/00; A61B 5/00; G01N 33/497

(52) U.S. Cl. CPC A61B 5/082; G01N 33/497; G01N 33/0047; A61B 5/486; A61B 5/7246; A61B 5/742; A61B 5/7275; A61B 5/6898; G01N 2033/4975

(57) ABSTRACT

A health support device 10 acquires a measured ketone concentration by measuring ketones excreted from a user, acquires a target curve that expresses changes in target ketone concentration corresponds to a health objective of the user, and outputs advice information corresponding to the health objective based on a comparison result between the ketone concentration and the target curve.

### CROSS-REFERENCE TO RELATED APPLICATION

This application is based on and claims the benefit of priority of the prior Japanese Patent Application No. 2013-262935, filed on Dec. 19, 2013, the entire contents of which are incorporated herein by reference.

### BACKGROUND

1. Technical Field

The present invention relates to a health support device, method, and recording medium stored with a program.

2. Related Art

In order to go on a diet healthily, there is a need to reduce the amount of body fat appropriately. In order to achieve this, there is a need to ascertain the state of fat burning, and to take exercise and food at appropriate timings.

It is already known that the state of fat burning can be ascertained by measuring the concentration of acetone in exhaled air. For example, in Patent Document 1, a diet support system is described that measures the concentration of acetone in exhaled air, and determines the appropriate timing to consume food, or the appropriate timing to take exercise, according to the results of analyzing the measured acetone concentration.

### PATENT DOCUMENTS

Patent Document 1: International Publication (WO) No. 2013/038959

However, in the above conventional technology, determination is simply made as to whether or not the measured acetone concentration is higher or lower than a predetermined threshold value, and the appropriate timing to consume food, or the appropriate timing to take exercise, is determined based on this determination result, and there is no mention of a subsequent action plan. It is accordingly not always capable of supporting slimming appropriately.

Sometimes, in order to maintain health, there is an objective of achieving a healthy increase in body fat rather than slimming. However, in the conventional technology, due to the simplistic determination of being higher or lower than the predetermined threshold value, it is not possible to determine the appropriate timing to consume food, or the appropriate timing to take exercise, corresponding to various health objectives. For example, it is not possible to determine with good precision whether or not slimming goes well as planned. There is accordingly a concern that excessive slimming may be performed, such as abstaining from three meals.

### SUMMARY

An object of the present invention is to provide a health support device, a method, and a program stored on a recording medium, that are capable of appropriately supporting action corresponding to a health objective.

In order to solve the above issues, a health support device of a first aspect of the present invention includes a ketone concentration acquisition section that acquires a measured ketone concentration by measuring ketones excreted from a user, a target curve acquisition section that acquires a target curve expressing changes in target ketone concentration corresponding to a health objective of the user, and an output section that outputs advice information corresponding to the health objective based on a comparison result between the measured ketone concentration and the ketone concentration of on the target curve.

As in a second aspect, configuration may be made such that the output section outputs the advice information corresponding to the health objective based on comparison results between plural measured ketone concentrations in the same day, and plural target ketone concentrations on the target curve that correspond to respective measurement times of the plural measured ketone concentrations.

As in a third aspect, configuration may be made such that the output section calculates a measured ketone concentration integral value based on a concentration difference between the plural measured ketone concentrations in the same day and a time difference between measurement times, and calculates a target ketone concentration integral value based on concentration differences between the plural target ketone concentrations and time differences between the measurement times, and outputs the advice information corresponding to the health objective based on a comparison result between the measured ketone concentration integral value and the target ketone concentration integral value.

As in a fourth aspect, configuration may be made such that in cases in which the slope of a line connecting the plural measured ketone concentrations is downward and the measured ketone concentration integral value is smaller than the target ketone concentration integral value, the output section outputs advice information related to at least one result out of eating or exercise if the difference between the measured ketone concentration integral value and the target ketone concentration integral value is a predetermined threshold value or greater.

As in a fifth aspect, configuration may be made such that in cases in which the slope of a line connecting the plural measured ketone concentrations is upward and the measured ketone concentration integral value is greater than the target ketone concentration integral value, the output section outputs advice information related to at least one result out of eating or exercise if the difference between the measured ketone concentration integral value and the target ketone concentration integral value is a predetermined threshold value or greater.

As in a sixth aspect, configuration may be made such that the output section derives an eating time based on an actual measurement curve expressing the plural measured ketone concentrations, and outputs advice information related to eating time based on the derived eating time and an eating time on the target curve.

As in a seventh aspect, configuration may be made such that the output section outputs achievement points derived based on a difference between, or a ratio of, the measured ketone concentration and the target ketone concentration.

As in an eighth aspect, configuration may be made such that the output section calculates an overall achievement points based on plural instances of achievement points derived for plural measured ketone concentrations measured in the same day, and outputs the calculated overall achievement points.

As in a ninth aspect, configuration may be made such that the output section outputs an action plan screen image expressing an action plan corresponding to the target curve for each respective period of time.

As in a tenth aspect, configuration may be made such that the output section outputs a concentration change screen image that displays the actual measurement curve representing changes in the measured ketone concentration and the target curve.

As in an eleventh aspect, configuration may further include a measurement section that measures ketones excreted from a user.

As in a twelfth aspect, configuration such that the ketones excreted from the user are acetone contained in breath exhaled from the user is preferable.

A thirteenth aspect of the present invention is a health support method including acquiring a measured ketone concentration of measured ketones excreted from a user, acquiring a target curve expressing changes in target ketone concentration corresponding to a health objective of the user, and outputting advice information corresponding to the health objective based on a comparison result between the measured ketone concentration and the target curve.

A fourteenth aspect of the present invention is a non-transitory recording medium stored with a health support program that causes processing to be executed on a computer, the processing including acquiring a measured ketone concentration by measuring ketones excreted from a user, acquiring a target curve expressing changes in target ketone concentration corresponding to a health objective of the user, and outputting advice information corresponding to the health objective based on a comparison result between the measured ketone concentration and the target curve.

### Advantageous Effects of Invention

According to the present invention, the advantageous effect is exhibited of being capable of appropriately supporting action corresponding to a health objective.

### BRIEF DESCRIPTION OF DRAWINGS

FIG. 1 is a diagram of the external appearance of a health support device.

FIG. 2 is a block diagram of a health support device.

FIG. 3 is a flowchart of processing by a health support program.

FIG. 4 is a diagram illustrating an example of a health objective registering screen image.

FIG. 5 is a graph illustrating an example of a target curve when the health objective is “slimming”.

FIG. 6 is a graph illustrating an example of an ideal curve when the health objective is “health improvement”.

FIG. 7 is a graph illustrating an example of an ideal curve when the health objective is “maintenance of health”.

FIG. 8 is a graph illustrating an example of an ideal curve when the health objective is “healthy weight gain”.

FIG. 9 is a diagram illustrating an example of table data representing an ideal curve.

FIG. 10 is a diagram illustrating an example of table data representing an ideal curve.

FIG. 11A is a diagram illustrating an example of an advice table when the health objective is “slimming”.

FIG. 11B is a diagram illustrating an example of an advice table when the health objective is “health improvement”.

FIG. 11C is a diagram illustrating an example of an advice table when the health objective is “maintenance of health”.

FIG. 11D is a diagram illustrating an example of an advice table when the health objective is “healthy weight gain”.

FIG. 12 is a diagram illustrating an example of an action plan screen image.

FIG. 13 is a diagram illustrating an example of a measured acetone concentration display screen image.

FIG. 14 is a diagram illustrating an example of an achievement point table.

FIG. 15 is a diagram illustrating an example of an achievement point display screen image.

FIG. 16 is a diagram illustrating an example of a concentration change screen image.

FIG. 17 is a diagram to explain an evaluation method of deviation from an ideal curve.

FIG. 18 is a diagram to explain an evaluation method of deviation from an ideal curve.

FIG. 19 is a diagram to explain an evaluation method of deviation from an ideal curve.

FIG. 20 is a table illustrating an example of an advice table.

FIG. 21 is a diagram illustrating an example of an acetone data display screen image.

FIG. 22 is a table illustrating an example of an advice table.

FIG. 23 is a block diagram of a mode of connection between a measurement device and a personal computer.

### DESCRIPTION OF EMBODIMENTS

Explanation follows regarding an exemplary embodiment of the present invention.

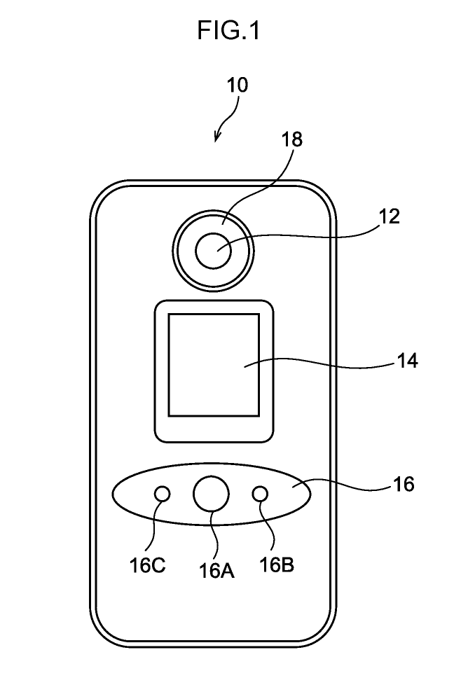

FIG. 1 is a diagram of the external appearance of a health support device 10 according to the present exemplary embodiment. As illustrated in FIG. 1, the health support device 10 includes a measurement section 12, a display section 14, and an operation section 16. The health support device 10 according to the present exemplary embodiment is, as an example, a portable device that is convenient to be carried around.

The measurement section 12 measures the concentration of ketones excreted by a user (referred to below as ketone concentration). Ketone is a collective name employed for acetoacetic acid, 3-hydroxy acetic acid (β-hydroxy acetic acid), and acetone, and represents at least one thereof.

In the present exemplary embodiment, explanation is given of a case in which the measurement section 12 is, as an example, configured with an acetone detection sensor that detects acetone in the breath of a user. The user is able to measure the acetone concentration of exhaled breath by blowing air into a blow hole 18.

The display section 14 is configured, for example, by a liquid crystal panel or the like. Detailed description is given later, however, briefly, various screen images are displayed on the display section 14, such as, for example, various setting screen images, the measurement results of acetone concentration measured by the measurement section 12, and advice information based on the measured acetone concentration. The display section 14 may also be configured including a touch panel function, and may be configured to enable operation by directly touching the screen.

The operation section 16 is configured by including plural operation buttons, and FIG. 1 illustrates an example of a case in which there are 3 individual operation buttons 16A to 16C provided.

The operation button 16A functions, as an example, as a button to operate to switch the power source of the health support device 10 ON/OFF, and to make selections on various screen images.

The operation button 16B functions, as an example, as a button to input data on various screen images.

The operation section 16C functions, as an example, as a button to instruct reading of past measurement results and the like.

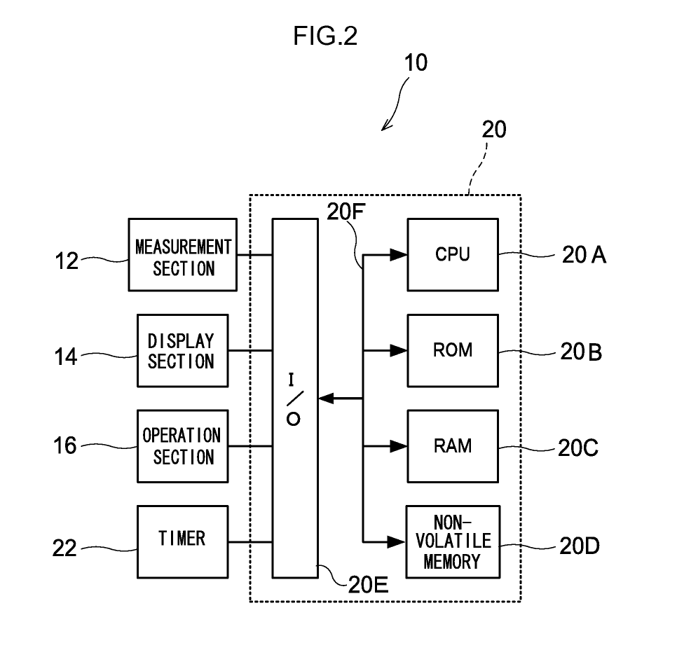

FIG. 2 is a block diagram of the health support device 10. As illustrated in FIG. 2, the health support device 10 includes a controller 20. The controller 20 is configured by including a Central Processing Unit (CPU) 20A, Read Only Memory (ROM) 20B, Random Access Memory (RAM) 20C, non-volatile memory 20D, and an input-output (I/O) interface 20E, each connected together through a bus 20F. In this case, a health support program for executing health support processing, explained later, in the CPU 20A of the controller 20 is, for example, pre-written to the non-volatile memory 20D, and read into and executed by the CPU 20A. The health support program may be provided by storing on a recording medium, such as a CD-ROM, memory card, or the like, or may be downloaded from a server, not illustrated in the drawings.

The measurement section 12, the display section 14, the operation section 16, and a timer 22 are connected to the I/O interface 20E. The timer 22 includes a time acquisition function to acquire the current time, and to clock a set period of time.

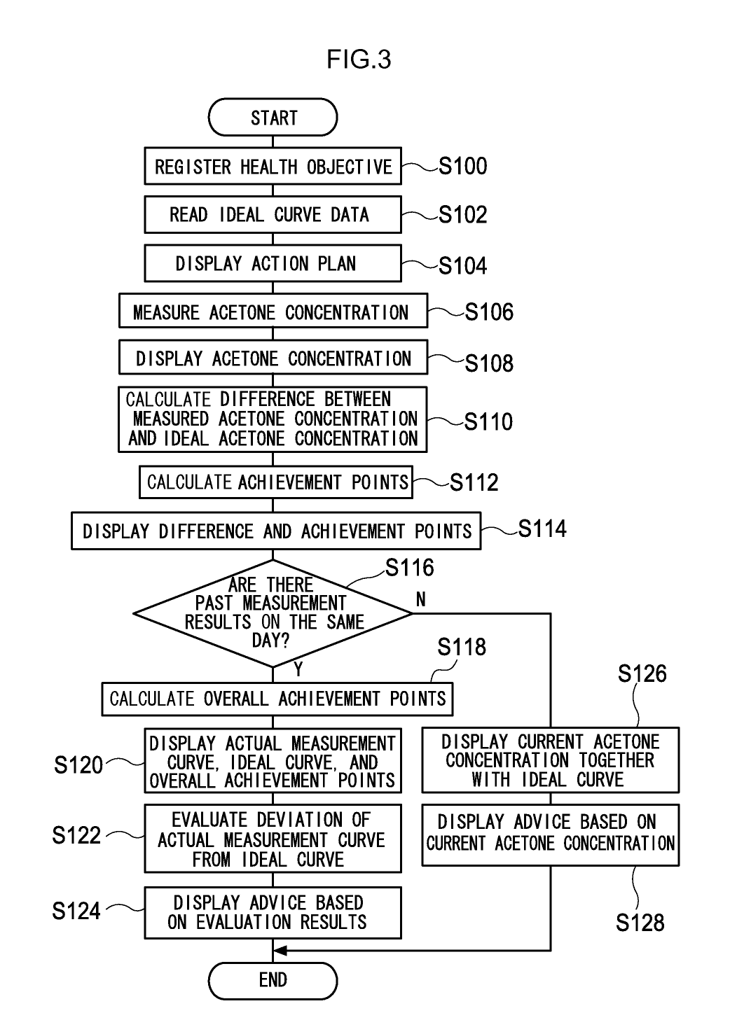

Explanation next follows, as operation of the present exemplary embodiment, regarding processing by the health support program executed by the CPU 20A of the controller 20, with reference to the flowchart illustrated in FIG. 3. The processing in FIG. 3 is executed when a user operates the operation section 16 of the health support device 10, and instructs execution of the health support program.

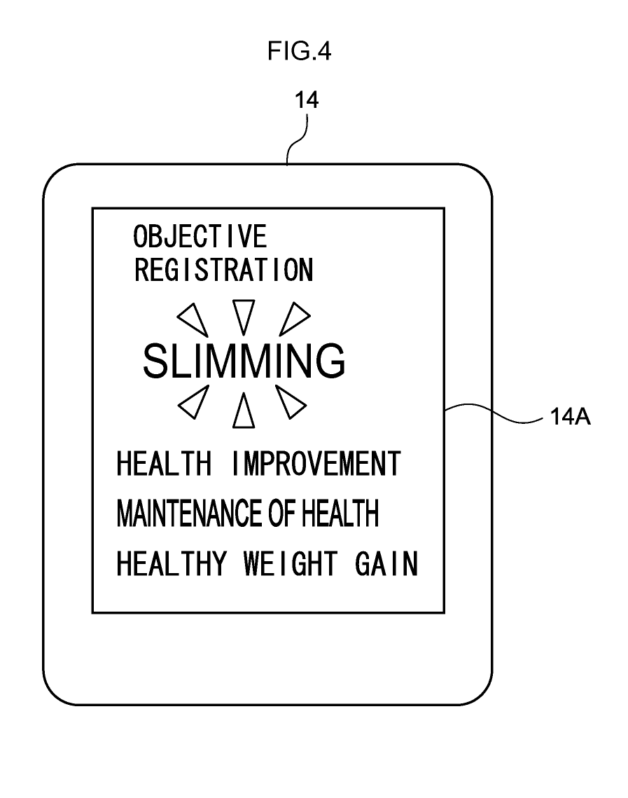

First, at step S100, a user registers a health objective. More specifically, a health objective registering screen image 14A such as that illustrated in FIG. 4 is displayed on the display section 14.

Plural health objectives are displayed on the health objective registering screen image 14A. In the present exemplary embodiment, as an example, there are four health objectives displayed, these being “slimming”, “health improvement”, “maintenance of health”, and “healthy weight gain”; however, health objectives are not limited thereto. The user operates the operation section 16 and selects a desired health objective from out of the plural health objectives being displayed. When the health objective has been selected by the user, the CPU 20A stores the selected health objective in the non-volatile memory 20D as a registered health objective.

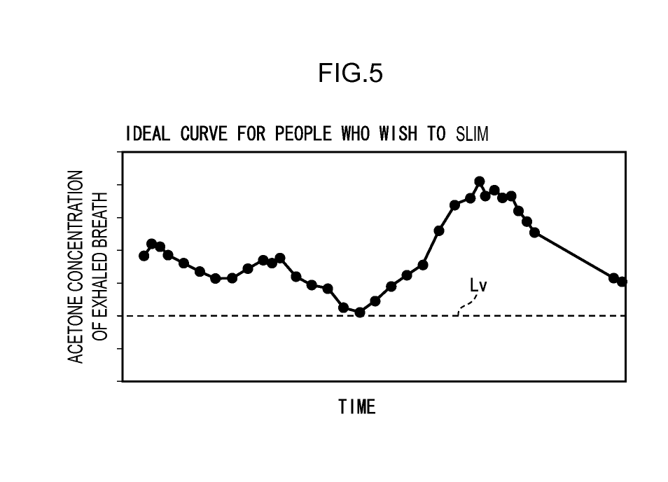

At step S102, a target curve corresponding to the registered health objective is read from the non-volatile memory 20D. The target curve is a curve expressing changes in acetone concentration to achieve the health objective. In the present exemplary embodiment, explanation follows regarding a case in which the target curve is an ideal curve expressing ideal changes in acetone concentration to achieve the health objective. FIG. 5 illustrates an example of an ideal curve when the health objective is “slimming”. As illustrated in FIG. 5, the ideal curve is a curve expressing a correspondence relationship of time, in a predetermined period of time during a day (for example the period of time when awake), against acetone concentration of exhaled breath. The ideal curve illustrates the changes in the acetone concentration during this period of time.

As illustrated in FIG. 5, the ideal curve when the health objective is “slimming” is one in which the acetone concentration is equal to or higher than a predetermined level Lv through all the predetermined periods of time, and is a curve in which the acetone concentration tends to be high throughout the day, namely fat is burned and much acetone is excreted, enabling slimming.

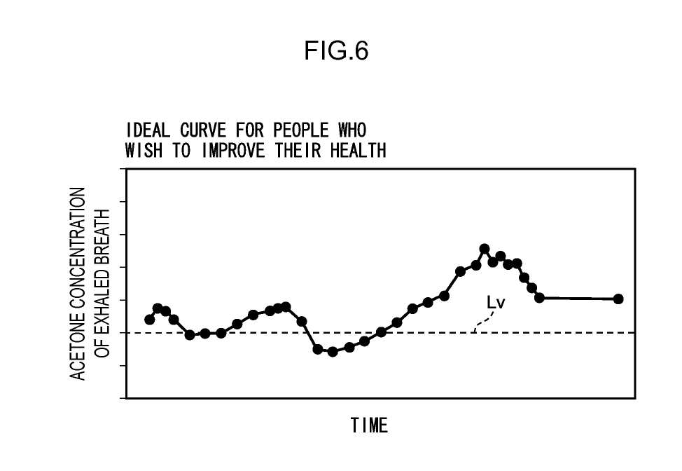

FIG. 6 illustrates an example of an ideal curve when the health objective is “health improvement”. The ideal curve when the health objective is “health improvement” is one in which the acetone concentration is equal to or higher than the predetermined level Lv through most of the periods of time, and is a curve in which the acetone concentration tends to be high throughout the day. Rises in blood sugar after eating are not liable to occur and rapid rises in insulin secretion are suppressed by the small decrease in acetone concentration after eating, and quickly reversing into a rise, and so is a curve that enables a health improvement to be achieved by reducing damage to blood vessels.

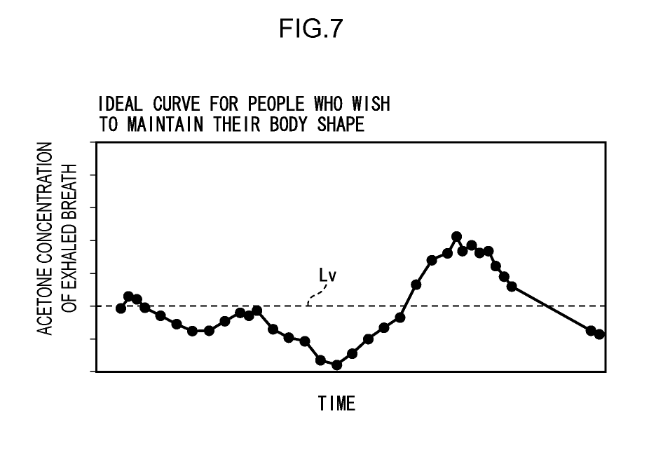

FIG. 7 illustrates an example of an ideal curve when the health objective is “maintenance of health”, namely for people who wish to maintain their body shape. The ideal curve when the health objective is “maintenance of health” is one in which the acetone concentration is less than the predetermined level Lv over most of the periods of time, but also has periods of time when the acetone concentration is equal to or greater than the predetermined level Lv, and is a curve that enables body shape to be maintained.

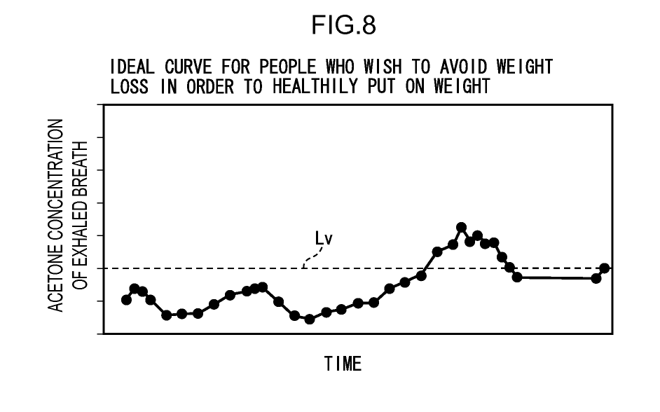

FIG. 8 illustrates an example of an ideal curve when the health objective is “healthy weight gain”, namely for people who wish to avoid weight loss in order to healthily put on weight. The ideal curve for the health objective “healthy weight gain” is one in which the acetone concentration is less than the predetermined level Lv over most of the periods of time, also having even more periods of time when the acetone concentration is less than the predetermined level Lv than for the health objective of “maintenance of health”, and is a curve that also has periods of time when the acetone concentration is equal to or greater than the predetermined level Lv. Rises in blood sugar after eating are not liable to occur and rapid rises in insulin secretion are suppressed by the small decrease in acetone concentration after eating, and quickly reversing into a rise, and so this is a curve that enables a healthy weight gain to be achieved by reducing damage to blood vessels.

In the present exemplary embodiment, the ideal curves corresponding to the four health objectives listed above are pre-stored in the non-volatile memory 20D; however, the ideal curves are not limited to the four described above.

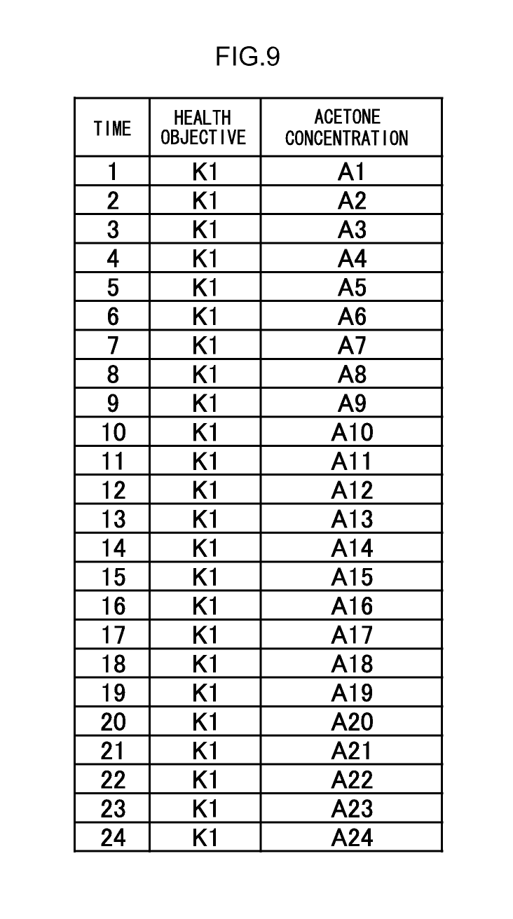

Each of the ideal curves may, as illustrated in FIG. 9, be derived from table data expressing correspondence relationships between time, health objective, and acetone concentration, and may be expressed by an equation.

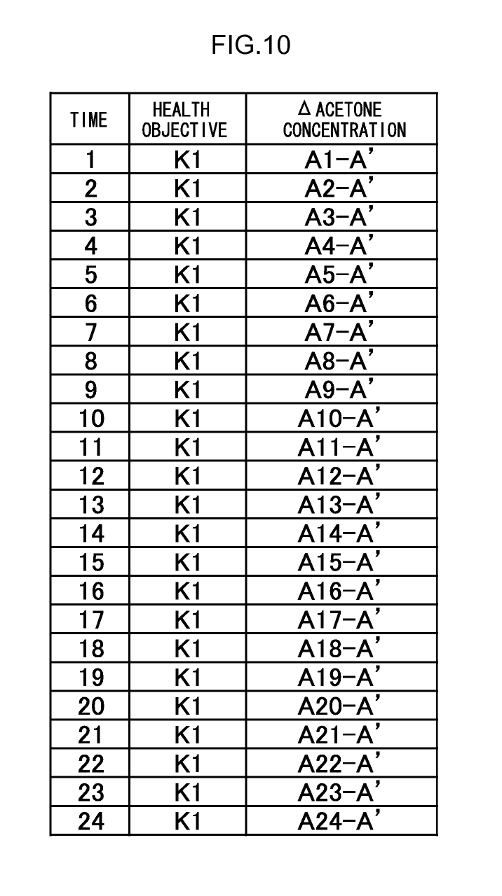

The table data illustrated in FIG. 9 is, for example, statistically derived based on pre-measured results of acetone concentration at every period of time for example every hour for many users. It may also be statistically derived by regression analysis based on pre-measured results of acetone concentration at every period of time for many users, as a regression equation expressing a relationship between time and acetone concentration. The regression equations are different for each of the health objectives, and, with parameters of coefficient(s) and time, may be a polynomial equation (an n-order equation of time t), or may be a first order equation or quadratic equation, or may be an equation including an inverse, an index calculation, or a logarithmic calculation. Each of the ideal curves may, as illustrated in FIG. 10, be derived from table data expressing correspondence relationships between time, health objective, and Δ acetone concentration. Δ acetone concentration at every period of time illustrated in FIG. 10 is data representing the difference (acetone concentration change amount) between the acetone concentration At and a predetermined reference acetone concentration A′ at time t. Table data expressing a correspondence relationship between time, health objective, and acetone concentration integration values may also be employed.

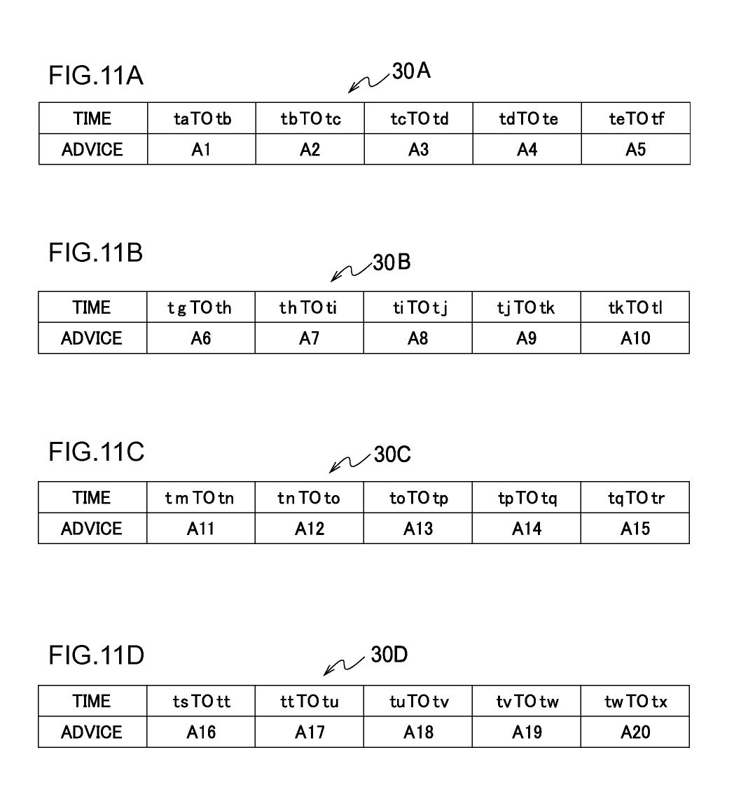

At step S104, an action plan screen image is generated indicating an action plan corresponding to the registered health objective, based on the ideal curve read at step S102, and the advice table as illustrated in FIGS. 11A to 11D pre-stored in the non-volatile memory 20D.

FIG. 11A illustrates an advice table 30A when the health objective is “slimming”. As illustrated in FIG. 11A, the advice table 30A is data expressing a correspondence relationship between divided period of time (ta to tb, tb to tc, and so on up to te to tf), that are a predetermined period of time (the period of time when awake) divided into plural period of time, and advice (A1, A2, and so on up to A5). The advice here refers to what action should be performed in the corresponding period of time, namely an action plan.

FIG. 11B illustrates an advice table 30B when the health objective is “health improvement”, FIG. 11C illustrates an advice table 30C when the health objective is “maintenance of health”, and FIG. 11D illustrates an advice table 30D when the health objective is “healthy weight gain”. Due to the ideal curve being different for each of the health objectives, as illustrated in FIG. 11A to 11D, the periods of time and advice in the advice tables 30A to 30D are also different for each of the health objectives. Each of the periods of time may be a few hours, or may be a few tens of minutes, and the manner of division is not limited.

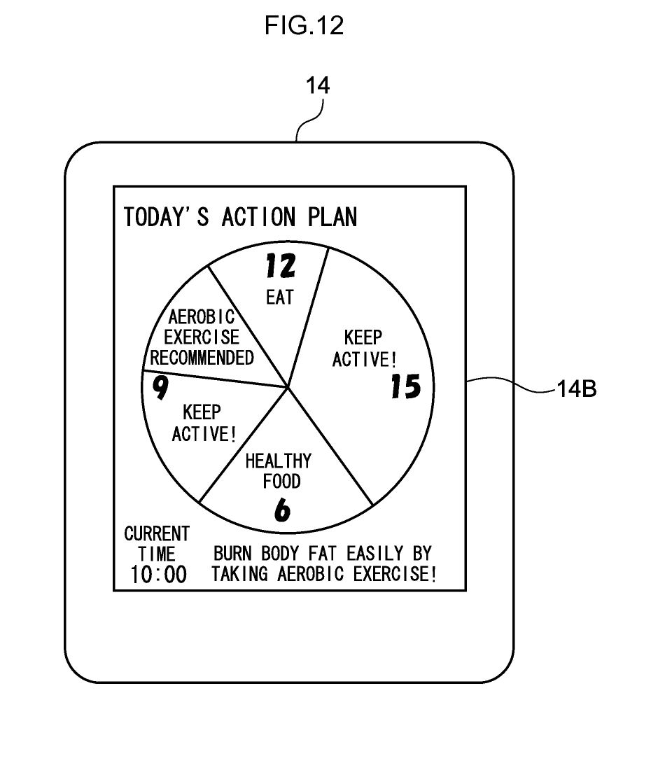

Based on the advice tables corresponding to such health objectives, for example, an action plan screen image 14B, such as illustrated in FIG. 12, is generated and displayed on the display section 14. The action plan screen image 14B expresses, as an example, 12 hours' worth of advice, starting from the current time, when the health objective is “slimming”.

In the example in FIG. 12, the advice in the period of time from the current time of 10 o'clock to 11 o'clock is “aerobic exercise recommended”, the advice in the period of time 11 o'clock to 13 o'clock is “eat”, the advice of the period of time from 13 o'clock to 17 o'clock is “keep active!”, the advice in the period of time 17 o'clock to 19 o'clock is “healthy food”, and the advice in the period of time 19 o'clock to 22 o'clock is “keep active!”; however there is no limitation thereto.

In this manner, generating and displaying the action plan screen image corresponding to the health objective on the display section 14 enables the user to readily ascertain what action should be performed to enable the desired health objective to be achieved.

At step S106, the acetone concentration is measured. More specifically, first, a message is displayed on the display section 14 that measurement of acetone concentration will start after a predetermined time (for example 10 seconds) has elapsed, and the timer 22 is instructed to count the predetermined time.

Then, when notified by the timer 22 that the predetermined time has elapsed, a blowing start message is displayed on the display section 14 instructing air to be blown in through the blow hole 18, and the measurement section 12 is instructed to start measuring the acetone concentration. The user blows air into the blow hole 18 when the blowing start message is displayed on the display section 14.

The measurement section 12 measures the acetone concentration of air that has been blown into the blow hole 18, and outputs the measurement to the controller 20. The measured acetone concentration is stored in the non-volatile memory 20D together with the current time acquired from the timer 22.

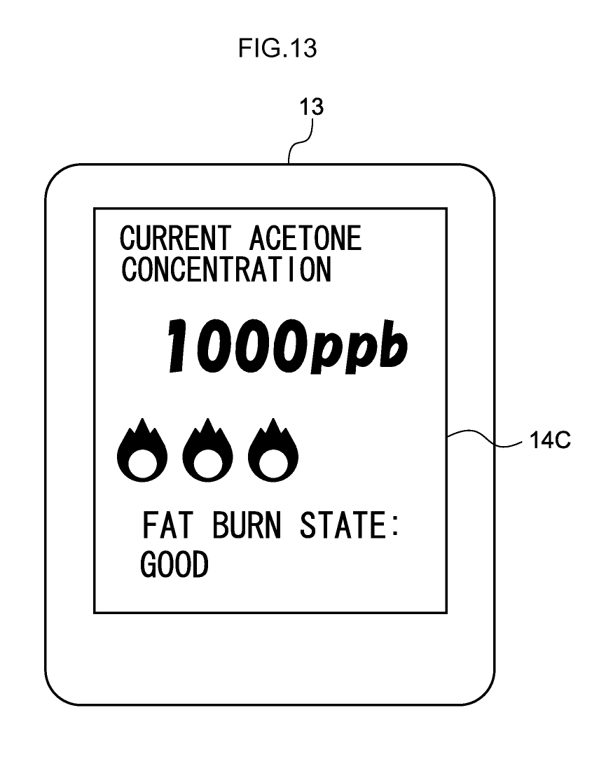

At step S108, the acetone concentration measured at step S106 (referred to below as the measured acetone concentration) is displayed on the display section 14. FIG. 13 illustrates an example of a measured acetone concentration display screen image. As illustrated in FIG. 13, as an example, the current acetone concentration and the fat burn state corresponding to the current acetone concentration are displayed on the measured acetone concentration display screen image 14C. The fat burn state is, as an example, expressed by a fire symbol and text, and a greater number of fires indicates that more fat is being burned. The number of fires displayed is determined so as to increase as the measured acetone concentration increases. The fat burn state may be displayed by a symbol other than that of a fire.

At step S110, the difference is calculated between the measured acetone concentration (referred to below as the measured acetone concentration Conc1), and the acetone concentration corresponding to the current time on the ideal curve acquired at step S102 (referred to below as the ideal acetone concentration Conc2). More specifically, taking the current time as t1, where t=t1, then the ideal acetone concentration Conc2 is calculated from table data such as that illustrated in FIG. 9, or a regression equation, corresponding to the ideal curve associated with the registered health objective. The difference Δ Conc between the calculated ideal acetone concentration Conc2 and the measured acetone concentration Conc1 is calculated according to the following equation.

ΔConc=Conc2−Conc1 Equation (1)

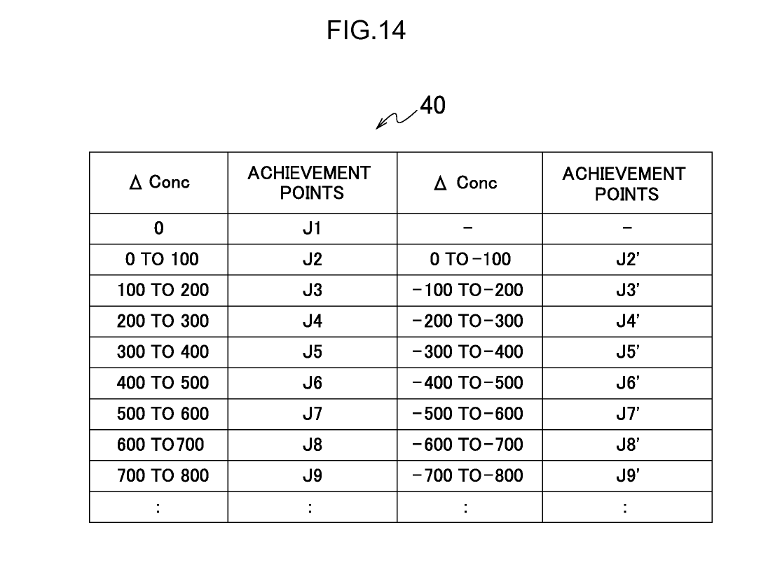

At step S112, achievement points corresponding to the difference Δ Conc calculated at step S110 are derived. The achievement points are, more specifically, as illustrated in FIG. 14, derived from an achievement point table 40 expressing a correspondence relationship between the difference Δ Conc and the achievement points. The achievement point table 40 is pre-stored in the non-volatile memory 20D. The derived achievement points are stored in the non-volatile memory 20D together with the current time.

The achievement points are, for example, set at the highest value when the difference Δ Conc is 0, and at values that get smaller the greater the absolute value of the difference Δ Conc. The greater the achievement points, the nearer to the ideal acetone concentration. Namely, it could be said that the achievement points indicate the degree of target achievement.

Moreover, Equation (1) is an equation to calculate the difference between the ideal acetone concentration Conc2 and the measured acetone concentration Conc1; however, configuration may be made such that a ratio (for example Conc2/Conc1) is derived. In such cases, the closer the ratio to 1, the closer to the ideal. Accordingly, the achievement points may be set to be greater the closer the ratio is to 1.

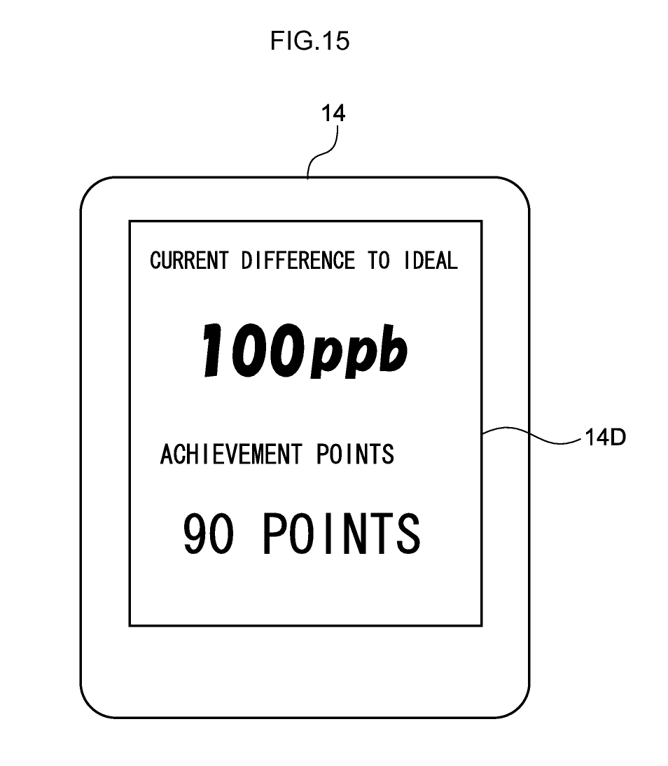

At step S114, the difference Δ Conc calculated at step S110 and the achievement points derived at step S112 are displayed on the display section 14. FIG. 15 illustrates an example of an achievement point display screen image displaying the difference Δ Conc and the achievement points. As illustrated in FIG. 15, the achievement point display screen image 14D is displayed with the difference Δ Conc displayed as “current difference to ideal”, together with the achievement points. The display method is not limited thereto.

At step S116, determination is made as to whether or not past calculated achievement points on the same day are stored in the non-volatile memory 20D, and processing proceeds to step S118 if stored, and processing proceeds to step S126 if not stored.

At step S118, the overall achievement points for acetone concentration on that day are calculated. More specifically, an overall achievement points Pall is calculated according to the following equation, wherein there are n individual (positive number) instances of calculated achievement points P calculated for the same day, denoted P1 to Pn.

Pall=(P1+P2+ and so on up to Pn)/n Equation (2)

Namely, the overall achievement points Pall is the average value of the achievement points for the measured acetone concentration during the same day, with the higher the value thereof indicating the smaller the difference to the ideal, and the higher the degree of achievement of the registered health objective. The calculation method of the overall achievement points Pall is not limited thereto. For example, Equation (2) is an equation employing a sum and a division, however it may be an equation employing a difference and an integral, as long as it is an equation expressing higher overall achievement points the higher the degree of achievement of the registered health objective.

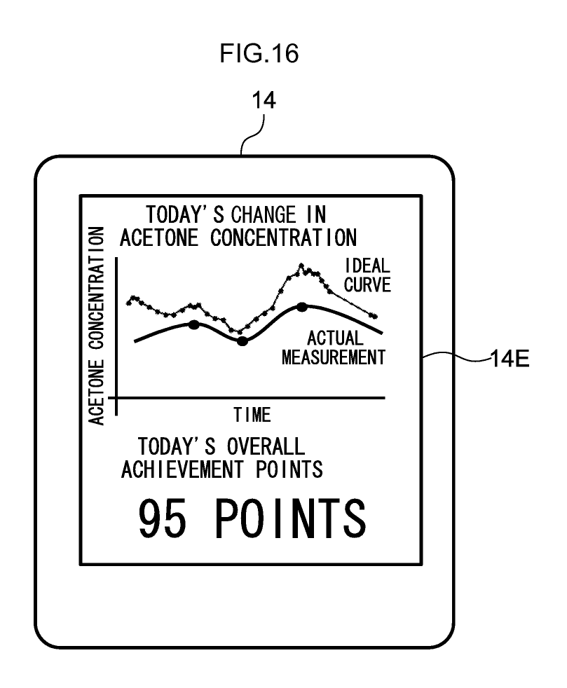

At step S120, as illustrated in FIG. 16, a concentration change screen image 14E, displaying the overall achievement points calculated at step S118, the ideal curve read at step S102, and the actual measurement curve stored in the non-volatile memory 20D expressing the changes in measured acetone concentration on the same day, is displayed on the display section 14.

At step S122, the deviation of the actual measurement curve from the ideal curve is evaluated based on the difference between the actual measurement curve and the ideal curve. The acetone concentration is lower when there is excessive carbohydrate energy in the body, since fat is not burned, and fat burning is higher when carbohydrate energy in the body is insufficient. Thus normally after eating there is excessive carbohydrate energy in the body, the acetone concentration starts to fall, and the acetone concentration subsequently rises when carbohydrate energy no longer suffices. Thus, since normally food is consumed three times a day, the ideal curve of acetone concentration may be thought of as having three peaks and three troughs. The peaks in acetone concentration are when the acetone concentration gradually transitions from upward to downward. In contrast thereto, troughs in acetone concentration are when the acetone concentration gradually transitions from downward to upward.

Various evaluation methods may be considered as a method for evaluating deviation of the actual measurement curve from the ideal curve. Explanation first follows regarding a first evaluation method.

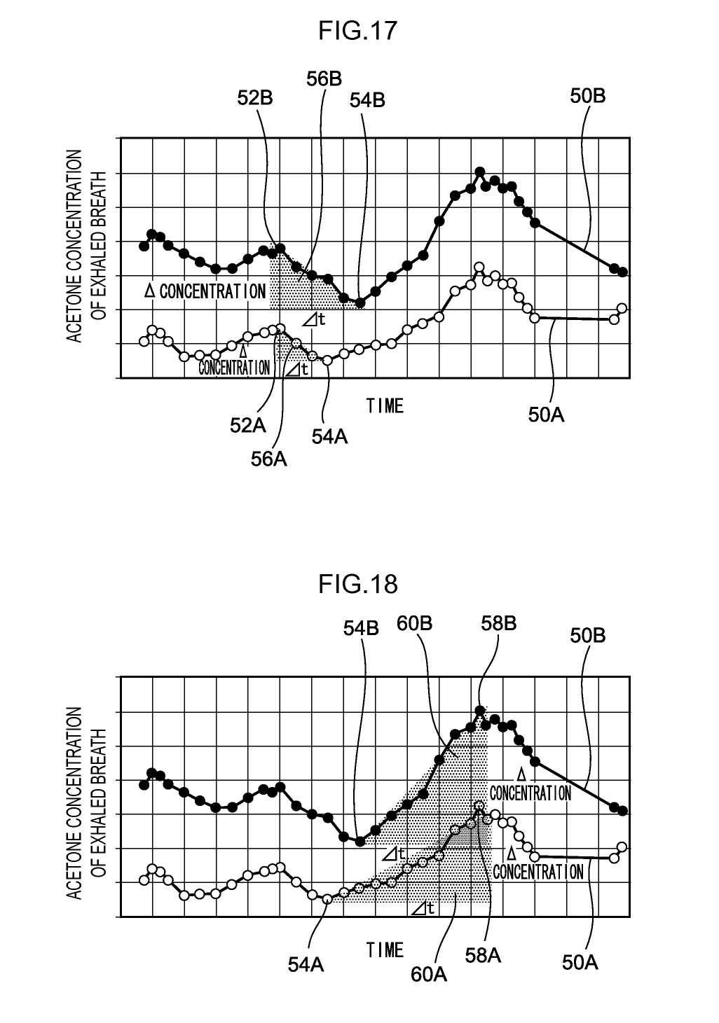

In the first evaluation method, as, for example, illustrated in FIG. 17, an acetone concentration integral value D is calculated based on the concentration difference between a peak 52A and a trough 54A of an actual measurement curve 50A (Δ concentration), and the time difference from the peak 52A to the trough 54A (Δ t).

D=(Δconcentration×Δt)/2 Equation (3)

Namely, Equation (3) is an equation for calculating the surface area of a region 56A illustrated by hatching in FIG. 17. Equation (3) is an equation capable of simply deriving the acetone concentration integral value D, however the acetone concentration integral value D from the peak 52A to the trough 54A may be calculated by the following equation, wherein t1 is the time at peak 52A, t2 is the time at trough 54A, and Conc=f(t). This thereby enables a more accurate acetone concentration integral value D to be calculated.

D=∫ₜ₁^{t2}f(t)dt Equation (4)

Moreover, similarly for an ideal curve 50B, the acetone concentration integral value D is calculated by Equation (3) or Equation (4), based on the concentration difference (Δ concentration) between a peak 52B corresponding to the peak 52A of the actual measurement curve 50A and a trough 54B corresponding to the trough 54A of the actual measurement curve, and a time difference (Δ t) from the peak 52B to the trough 54B. Namely, the surface area of a region 56B illustrated by hatching in FIG. 17 is calculated. In the following, the calculated acetone concentration integral value for the region 56A of the actual measurement curve 50A is denoted DA, and the calculated acetone concentration integral value for the region 56B of the ideal curve 50B is denoted DB.

The acetone concentration integral values here may be considered as equivalent to the amount of fat burning. The regions 56A, 56B are regions where the acetone concentration falls from a peak to a trough, namely there is downward slope to the lines connecting the peaks to the troughs, and are regions where there is an excess of carbohydrate energy in the body after eating. The amount of fat burning is accordingly small when the acetone concentration integral value DA of the region 56A of the actual measurement curve 50A is smaller than the acetone concentration integral value DB of the region 56B of the ideal curve 50B, enabling determination that there is a small amount of fat burning, and that food has been consumed.

Thus in the first evaluation method, there is downward slope to the line connecting the peak to the trough, and if the acetone concentration integral value DA of the region 56A of the actual measurement curve 50A is smaller than the acetone concentration integral value DB of the region 56B of the ideal curve 50B, and that difference is equal to or greater than a predetermined threshold value, then evaluation is made that excessive food has been consumed, or insufficient exercise has been taken, or both. The threshold value is set at a value enabling determination when the value is equal to or greater than the threshold value, that excessive food has been consumed, or insufficient exercise has been taken, or both, and is itself determined from experimental results on many test subjects, or the like.

Explanation next follows regarding a second evaluation method. In the second evaluation method, for example as illustrated in FIG. 18, an acetone concentration integral value DA is calculated by Equation (3) or Equation (4), based on the concentration difference (A concentration) between the trough 54A and a peak 58A of the actual measurement curve 50A, and the time difference (A t) from the trough 54A to the peak 58A. Namely, the surface area of a region 60A illustrated by hatching in FIG. 18 is calculated.

Similarly for the ideal curve 50B, an acetone concentration integral value DB is calculated by Equation (3) or Equation (4), based on the concentration difference (Δ concentration) between a trough 54B corresponding to the trough 54A of the actual measurement curve 50A and a peak 58B corresponding to the peak 58A of the actual measurement curve, and the time difference (Δ t) from the trough 54B to the peak 58B. Namely, the surface area of a region 60B illustrated by hatching in FIG. 18 is calculated.

As stated above, the acetone concentration integral values here may be considered as equivalent to the amount of fat burning. The regions 60A, 60B are regions where the acetone concentration rises from a trough to a peak, namely there is upward slope to the lines connecting the troughs to the peaks, and are regions that transition to fat burning. The amount of fat burning is accordingly high when the acetone concentration integral value DA of the region 60A of the actual measurement curve 50A is larger than the acetone concentration integral value DB of the region 60B of the ideal curve 50B, enabling determination that there is a large amount of fat burning, and that exercise has been taken, or food has not been consumed, or both.

In the second evaluation method, there is upward slope to the line connecting the trough to the peak, and if the acetone concentration integral value DA of the region 60A of the actual measurement curve 50A is greater than the acetone concentration integral value DB of the region 60B of the ideal curve 50B, and that difference is equal to or greater than a predetermined threshold value, evaluation is made that exercise has been taken, or food has not been consumed, or both. The threshold value is set at a value enabling determination when the threshold value or greater that exercise has been taken, or food has not been consumed, or both, and is itself determined from experimental results on many test subjects, or the like.

In the first evaluation method and the second evaluation method, explanation has been given of a case in which deviation from the ideal curve is evaluated based on acetone concentrations of two points, that are a peak and a trough; however, a peak and a trough do not always have to be included, and evaluation of deviation from the ideal curve may be performed based on acetone concentrations of any freely selected two points. There is also no limit to two points, and evaluation of deviation from the ideal curve may be performed based on the acetone concentrations of any freely selected three points.

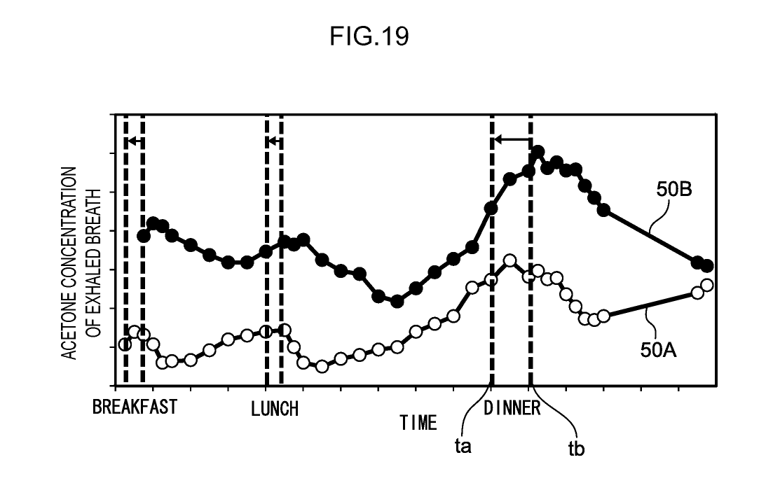

Explanation next follows regarding a third evaluation method. In the third evaluation method, for example, meal times are derived for each of the actual measurement curve 50A and the ideal curve 50B, and slippage of meal times is evaluated. As stated above, acetone concentration rises when carbohydrate energy in the body is insufficient, and acetone concentration starts to fall when food is consumed and there is excessive carbohydrate energy in the body. Thus it is conceivable that the meal time is a time just prior to the time when the acetone concentration curve starts to fall. The times just prior to the curves transitioning from rising to falling are accordingly derived as meal times for each of the actual measurement curve 50A and the ideal curve 50B, and the differences between the two times are derived for each meal (breakfast, lunch, dinner). Then, if the difference is equal to or greater than a predetermined threshold value, evaluation is made that meal times have skipped. For example, as illustrated in FIG. 19, if (tb−ta) is equal to or greater than a threshold value, wherein to is the time of dinner of the actual measurement curve 50A, and tb is the time of dinner of the ideal curve 50B, then evaluation is made that the meal time has skipped. The threshold value is set at a value enabling determination that a meal time has skipped if the value is equal to or greater than the threshold value, and is itself determined from experimental results on many test subjects, or the like. In cases in which meals are appropriately taken three times a day, at breakfast, lunch, and dinner, peaks should appear in the actual measurement curve 50A at three locations. However, if peaks appear at fewer than three locations, evaluation may be performed that a meal has been skipped, and if peaks appear at more than 3 locations, evaluation may be performed that snacking or eating between meals has consumed.

As described above, deviation from the ideal curve is evaluated based on at least one evaluation method from the first to the third evaluation methods, and stored in the non-volatile memory 20D together with the current time when evaluation was performed.

In the present exemplary embodiment, explanation has been given regarding the first to the third evaluation methods, however there is no limitation to these evaluation methods.

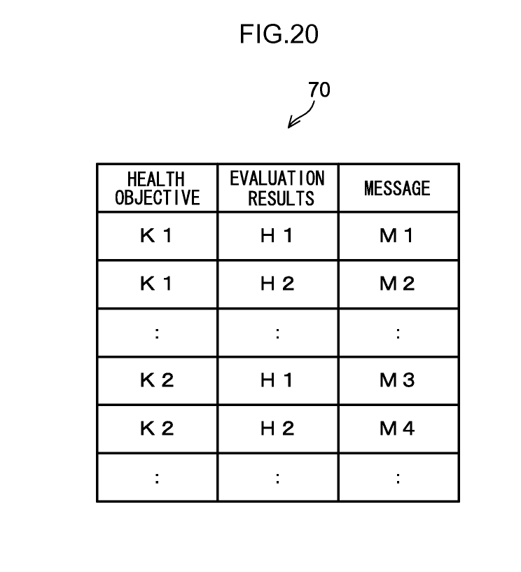

At step S124, advice information is displayed on the display section 14, based on the evaluation results at step S122. More specifically, an advice table 70 such as illustrated in FIG. 20 is stored in the non-volatile memory 20D, and a message derived from the advice table 70 is displayed as advice information on the display section 14. As illustrated in FIG. 20, the advice table 70 includes data representing health objectives, evaluation results, and messages, associated with each other. For example, if the health objective is “slimming”, and the evaluation result by the first evaluation method is “excessive food consumption”, then associated therewith as messages are, for example, messages recommending activity to promote fat burning, such as “You've been eating too many carbohydrates, haven't you?” and “Let's do some exercise!”. A message according to the evaluation result based on deviation from the ideal curve is accordingly displayed on the display section 14, enabling a user to readily ascertain the action they should adopt to achieve their health objective.

If determined at step S116 that there are no past calculated achievement points for the same day stored in the non-volatile memory 20D, processing proceeds to step S126.

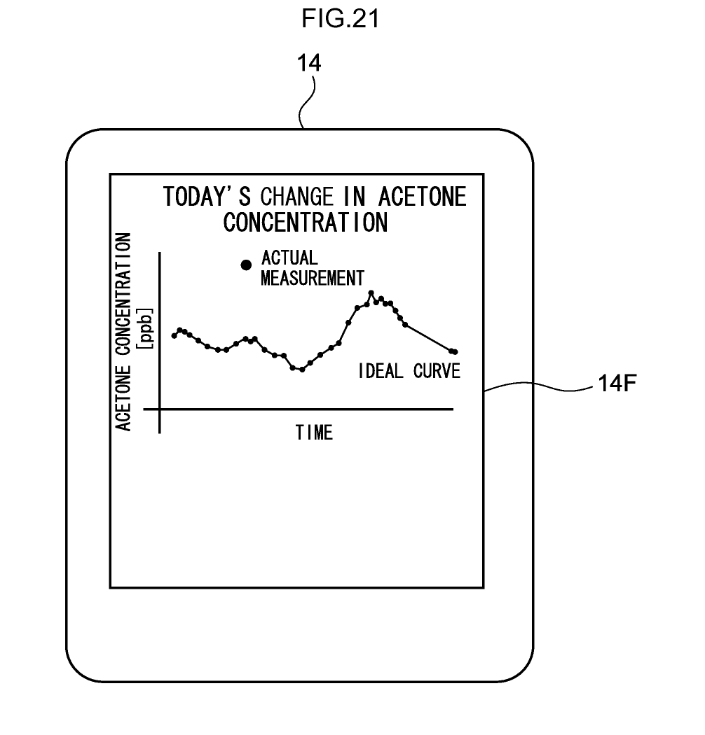

At step S126, the current acetone concentration is displayed on the display section 14 together with the ideal curve. FIG. 21 illustrates an example of an acetone concentration display screen image. As illustrated in FIG. 21, the current acetone concentration (actual measurement) is displayed on an acetone concentration display screen image 14F, together with the ideal curve. This thereby enables how much the current acetone concentration has deviated from the ideal to be readily ascertained.

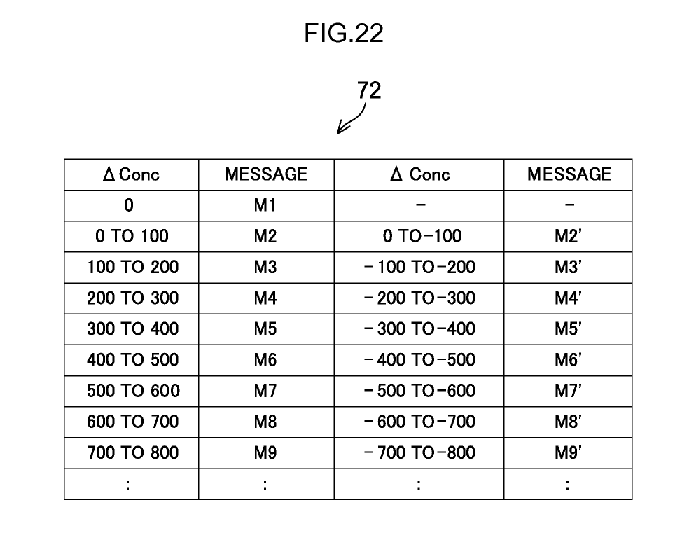

At step S128, advice information is displayed on the display section 14 based on the difference between the measured acetone concentration measured at step S106, and the ideal acetone concentration of the ideal curve at the time the measured acetone concentration was measured. For example, as illustrated in FIG. 22, an advice table 72 expressing correspondence relationships between messages and the difference Δ Conc between the measured acetone concentration and the ideal acetone concentration is pre-stored in the non-volatile memory 20D. A message corresponding to the difference between the measured acetone concentration and the ideal acetone concentration is then derived from the advice table 72, and displayed on the display section 14. This thereby enables a user to readily ascertain the action they should adopt to achieve their health objectives.

In this manner, in the present exemplary embodiment, ideal curves are stored in the non-volatile memory 20D for each of the health objective, deviation of the measured acetone concentration from the ideal curve is evaluated, and advice is given to the user for each of the health objective. This thereby enables the user to perform appropriate action corresponding to their health objectives, and, for example, excessive slimming, such as abstaining from all three meals, can be prevented, enabling a user to be appropriately supported to achieve their health objectives.

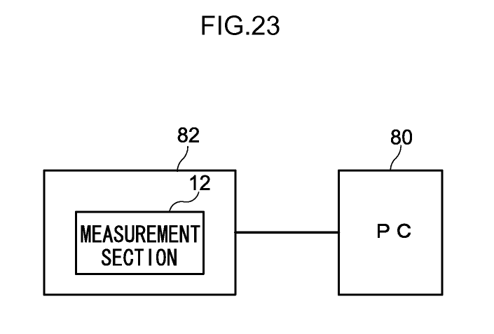

In the present exemplary embodiment, explanation has been given of a case in which the health support device 10 is a dedicated portable device; however, as illustrated in FIG. 23, configuration may be made by wired or wireless connection of a measurement instrument 82 including the measurement section 12 to a personal computer 80. In such cases, the personal computer 80 functions as the health support device by acquiring the acetone concentration measured by the measurement instrument 82 and executing the processing illustrated in FIG. 3.

The device connected to the measurement instrument 82 is moreover not limited to a personal computer, and a portable terminal such as a mobile phone, a smartphone, or a tablet may be employed. Such a portable terminal may be configured incorporating the measurement section 12. The portable terminal or the measurement instrument 82 may be configured with a network connection to a server. In such cases, the server functions as the health support device. Namely, the portable terminal or the measurement instrument 82 transmits the measured acetone concentration to the server, and the server executes the processing illustrated in FIG. 3 based on the acetone concentration received from the portable terminal or the measurement instrument 82, and transmits the result to the portable terminal or the measurement instrument 82. This thereby enables the measurement instrument 82 to be configured at low cost, sufficing with at least functionality to measure and transmit the acetone concentration to a server, and functionality to receive and display the result from the server.

In the present exemplary embodiment, explanation has been given of a case in which the measurement section 12 is configured with an acetone detection sensor that detects acetone in exhaled air; however there is no limitation thereto, and the measurement section 12 may be configured including a ketone detection sensor that detects ketones excreted from a user through skin, urine, saliva, or sweat.

In the present exemplary embodiment, explanation has been given of a case in which an ideal curve is pre-stored in the non-volatile memory 20D for each of the health objectives. However, for example, configuration may be made such that the ideal curves can be acquired by reading in, such as from a memory card, or configuration may be made such that the ideal curve is acquired from a server via a network through a wired or wireless connection, and then registered. Configuration may also be made such that a corrected ideal curve is acquired from a server and registered, or configuration may be made such that a user is able to correct the ideal curve.

In the present exemplary embodiment, explanation has been given in which ideal curves corresponding to the four individual health objectives of “slimming”, “health improvement”, “maintenance of health”, and “healthy weight gain”, are pre-stored in the non-volatile memory 20D; however, an ideal curve may be provided for each body weight and each body fat increase/decrease target, such as a target change amount in body weight (how much reduction or gain in body weight is desired), and a target change amount of body fat (how much reduction or gain in body fat is desired).

An ideal curve may also be provided for each lifestyle. For example, an ideal curve may be provided for each lifestyle of differing eating times for breakfast, lunch, and dinner, bedtimes, wake-up times, and preferable times for exercise. Configuration may then be made such that the lifestyle of a user is input, and deviation from an ideal curve is evaluated corresponding to the lifestyle input by the user.

Moreover, although explanation has been given in the present exemplary embodiment regarding a case in which the target curve is the ideal curve, configuration may be made, for example, to enable a target curve set by a user to be registered.

In the present exemplary embodiment, explanation has been given regarding evaluation of deviation between the actual measurement curve of the measured acetone concentration, and the ideal curve corresponding to the registered health objective; however, configuration may be made so that determination is made as to whether or not the actual measurement curve is close to one or other of the ideal curves in the non-volatile memory 20D, and the determination result displayed on the display section 14.

Moreover, configuration may be made such that user data is input, and the registered health objective and measurement data, including the acetone concentration measurement results and evaluation results, are stored in the non-volatile memory 20D for each user.

What is claimed is:

1. A health support device comprising:
a ketone concentration acquisition section that acquires a measured ketone concentration by measuring ketones excreted from a user;
a target curve acquisition section that acquires a target curve expressing changes in target ketone concentration corresponding to a health objective of the user; and
an output section that outputs advice information corresponding to the health objective based on a comparison result between the measured ketone concentration and the target curve.

2. The health support device of claim 1, wherein:
the output section outputs the advice information corresponding to the health objective based on comparison results between a plurality of measured ketone concentrations in the same day, and a plurality of target ketone concentrations on the target curve that correspond to respective measurement times of the plurality of measured ketone concentrations.

3. The health support device of claim 2, wherein:
the output section calculates a measured ketone concentration integral value based on a concentration difference between the plurality of measured ketone concentrations in the same day and a time difference between measurement times, and calculates a target ketone concentration integral value based on concentration differences between the plurality of target ketone concentrations and time differences between the measurement times, and outputs the advice information corresponding to the health objective based on a comparison result between the measured ketone concentration integral value and the target ketone concentration integral value.

4. The health support device of claim 3, wherein:
in cases in which the slope of a line connecting the plurality of measured ketone concentrations is downward and the measured ketone concentration integral value is smaller than the target ketone concentration integral value, the output section outputs advice information related to at least one result out of eating or exercise if the difference between the measured ketone concentration integral value and the target ketone concentration integral value is equal to or greater than a predetermined threshold value.

5. The health support device of claim 3, wherein:
in cases in which the slope of a line connecting the plurality of measured ketone concentrations is upward and the measured ketone concentration integral value is greater than the target ketone concentration integral value, the output section outputs advice information related to at least one result out of eating or exercise if the difference between the measured ketone concentration integral value and the target ketone concentration integral value is equal to or greater than a predetermined threshold value.

6. The health support device of claim 2, wherein:
the output section derives an eating time based on an actual measurement curve expressing the plurality of measured ketone concentrations, and outputs advice information related to eating time based on the derived eating time and an eating time on the target curve.

7. The health support device of claim 1, wherein:
the output section outputs achievement points derived based on a difference between, or a ratio of, the measured ketone concentration and the target ketone concentration.

8. The health support device of claim 7, wherein:
the output section calculates an overall achievement points based on a plurality of instances of achievement points derived for a plurality of measured ketone concentrations measured in the same day, and outputs the calculated overall achievement points.

9. The health support device of claim 1, wherein the output section outputs an action plan screen image expressing an action plan corresponding to the target curve for each respective period of time.

10. The health support device of claim 1, wherein the output section outputs a concentration change screen image that displays the actual measurement curve representing changes in the measured ketone concentration and the target curve.

11. The health support device of claim 1, further comprising:
a measurement section that measures ketones excreted from a user.

12. The health support device of claim 1, wherein the ketones excreted from the user are acetone contained in breath exhaled from the user.

13. A health support method comprising:
acquiring a measured ketone concentration of measured ketones excreted from a user;
acquiring a target curve expressing changes in target ketone concentration corresponding to a health objective of the user; and
outputting advice information corresponding to the health objective based on a comparison result between the measured ketone concentration and the target curve.

14. A non-transitory recording medium stored with a health support program that causes processing to be executed on a computer, the processing comprising:
acquiring a measured ketone concentration by measuring ketones excreted from a user;
acquiring a target curve expressing changes in target ketone concentration corresponding to a health objective of the user; and
outputting advice information corresponding to the health objective based on a comparison result between the measured ketone concentration and the target curve.
